(19) United States
(12) Patent Application Publication
(10) Pub. No.: US 2016/0022620 A1
(43) Pub. Date: Jan. 28, 2016

(54) ENCAPSULATED COMPOSITION FOR BINDING ALDEHYDES IN THE STOMACH

(71) Applicant: BIOHIT OYJ, Helsinki (FI)

(72) Inventor: Osmo SUOVANIEMI, Helsinki (FI)

(73) Assignee: BIOHIT OYJ, Helsinki (FI)

(21) Appl. No.: 14/774,868

(22) PCT Filed: Mar. 12, 2013
(86) PCT No.: PCT/FI2013/050275

§ 371 Date: Sep. 11, 2015

(51) Int. Cl. A61K 31/198; A61K 45/06; A61K 9/16

(52) U.S. Cl. CPC A61K 31/198; A61K 9/16; A61K 45/06; A61K 9/1617; A61K 9/1635; A61K 9/1641; A61K 9/1623; A61K 9/1611

(57) ABSTRACT

The present invention relates to non-toxic composition containing, as active compounds, one or more aldehyde-binding compounds, such as L- or D-cysteine, N-acetyl cysteine, and the pharmaceutically acceptable salts thereof, and optionally one or more further active compounds selected from sulphites and xylitol, the composition being used for decreasing the risk of a subject contracting cancer of the stomach, and indirectly of the small intestine and the large intestine. The composition is formulated into a controlled-release formulation consisting of granules contained in a capsule.

### FIELD OF THE INVENTION

The present invention relates to an encapsulated composition for effectively binding aldehydes in the stomach of a subject in order to decrease the risk of cancer in the stomach, intestine and/or colon of said subject. The invention relates also to methods for decreasing the risk of developing cancer in the gastrointestinal tract caused by aldehydes.

### DESCRIPTION OF RELATED ART

Both alcohol and smoking are risk factors for upper digestive tract cancers, and the combined use thereof multiplies the risk of developing an upper digestive tract cancer to as much as 150-fold (Salaspuro, 2003; and Francheschi et al. 1990).

The first metabolite of ethanol, acetaldehyde, is highly toxic, mutagenic and carcinogenic, as shown cell culture and animal experiments (IARC, 1999). Furthermore, epidemiological, genetic, microbiological and biochemical studies strongly suggest that acetaldehyde acts as a local and cumulative carcinogen in the upper digestive tract in humans (Salaspuro, 2009; Seitz and Stickel, 2010). Consequently, acetaldehyde present in alcoholic beverages and formed endogenously from ethanol was recently classified as carcinogenic to humans (group 1) by the International Agency for Research on Cancer (IARC) (Secretan et al. 2009).

Alcohol is evenly distributed in the liquid phase of the organs. Hence, after consuming alcohol, and as long as there is alcohol in the organs, the alcohol content in the blood, saliva, gastric juice and the contents of the intestine is the same. In that case, the microbes in the digestive tract are capable of oxidizing the alcohol to acetaldehyde. For example, even after a moderate dose of ethanol (0.5 g/kg), high acetaldehyde contents of a microbial origin (18-143 μM) have been found in human saliva; in other words, acetaldehyde builds up in saliva as an intermediate product of the microbial metabolism (Homann et al, 1997).

Acetaldehyde is also formed (particularly in the mouth, the pharynx, and the upper airways) as a consequence of smoking, and exposure to air contamination. It has been proven that chronic smoking significantly increases the acetaldehyde production of saliva originated in microbes. In fact, it has been demonstrated that the cancer risk associated with cigarette smoking is not only caused by the commonly known polycyclic aromatic hydrocarbons (PAH), but to a significant degree (up to 40%) by aldehydes, particularly by acetaldehyde and formaldehyde, whereas acrylic aldehyde causes up to 88.5% of the other toxic effects. Thus, a lowering of the aldehyde-content in cigarettes has been recommended (Haussmann, 2012).

The formation of acetaldehyde in the organism mainly takes place in the mouth, particularly in the saliva.

The average amount of saliva secreted by a human is 2.5 litres per day. The areas of influence of the acetaldehyde contained in the saliva include the mouth, the pharynx, the oesophagus and the stomach. Consequently, the effects of acetaldehyde may extend to the whole upper digestive tract area. On the other hand, carcinogenic acetaldehyde can be produced also endogenously by the oral microbes from various foodstuffs with high sugar or carbohydrate content, especially in an achlorhydric stomach. Atrophic gastritis and achlorhydria are well known risk factors of gastric cancer.

As a consequence of the microbial metabolism, acetaldehyde builds up in the stomach in the case, where the stomach is free from acid or has been made acid-free by medication. In the gastric juice there are for example Streptococcus viridans-bacteria, which have been shown to be excellent producers of acetaldehyde. Other effective acetaldehyde producers in acid-free stomach have been shown to be bacteria belonging to Neisseria, Rothia and Streptococcus salivarius (Väkeväinen et al., 2002).

Our recent studies show that in an achlorhydric stomach alcohol fermentation can start very quickly by the bacteria representing normal flora of the mouth or by yeasts present in the foodstuffs, for example by common baker's or brewer's yeast. These microbes can produce significant amounts of acetaldehyde and ethanol for example from carbohydrate containing foodstuffs, such as rice. This happens in particular, if the carbohydrate containing foodstuff is sweetened. For example in Asian countries the use of sweet sauces with rice is a very common practise. According to epidemiological studies the eating of rice causes a high risk for cancer in stomach.

In acid stomach the alcohol fermentation does not occur. On the other hand Helicobacter pylori infection and certain medicaments, such as Protein Pump Inhibitors (PPI) raise the pH of the stomach, whereby the same problem occurs.

One further risk factor for the stomach are foodstuffs comprising acetaldehyde. Our recent studies have shown that all sugar (saccharose, maltose, lactose) containing foodstuffs including beverages, can contain—or in the foodstuff is formed—significant amounts of acetaldehyde, 5 to 2000 μM and ethanol, 0.1 to 0.5 per mille. Some sour milks, yoghurts and juices contain acetaldehyde and ethanol as such (PCT/FI2006/000104).

It has also been shown that acetaldehyde builds up in the large intestine, as its bacteria that represent the normal flora are capable of converting ethanol into acetaldehyde (Jokelainen et al., 1996). In the intestines, endogenous ethanol can also be found, i.e. ethanol that is formed in the intestines in oxygen-free conditions under the effect of microbes. Acetaldehyde is formed, when this ethanol comes into contact with oxygen near the mucous membrane, for example.

The prior art discloses pharmaceutical compositions which contain compounds that bind acetaldehyde, their effect being based on the reaction of the effective substances with the acetaldehyde inside blood and/or cells, for example, U.S. Pat. No. 5,202,354, U.S. Pat. No. 4,496,548, U.S. Pat. No. 4,528,295, U.S. Pat. No. 5,922,346.

Acetaldehyde, which is formed in the organism when alcohol is consumed and thereafter, causes physiological symptoms called a hangover. Previously, efforts have been made to decrease the symptoms caused by acetaldehyde by taking preparations containing ascorbic acid, thiamine, cysteine or cysteic acid, and flavonoids or flavonoid complexes in a form of orally taken tablets in connection with, before or after consuming alcohol. When swallowed, the effective substances go to the stomach and small intestine and from there into the blood circulation (U.S. Pat. No. 5,202,354 and U.S. Pat. No. 4,496,548).

Suggestions have been made so as to use preparations containing amino acids, such as L-cysteine, methionine, taurine or arginine, ascorbic acid, vitamins A and E, which are sucked or chewed in the mouth, to reduce the effect of detrimental free radical compounds, which are formed when using tobacco products or being exposed to the same. It is believed that, after being absorbed, amino acids affect various tissues (U.S. Pat. No. 5,922,346; WO 99/00106).

WO 02/36098 suggests the use of compounds containing a free sulfhydryl and/or amino group for a local and long-term binding of acetaldehyde from saliva, the stomach or the large intestine. The compounds were mixed with a substance that enabled them to be released for at least 30 minutes in the conditions of the mouth, the stomach or the large intestine.

WO 2006/037848 suggests a composition comprising one or more free sulfhydryl and/or amino groups for removing or decreasing the aldehyde content of the saliva during smoking.

As on the basis of our recent studies, aldehydes play a considerable part in the pathogenesis of the stomach cancers, in particular by people having an achlorhydric stomach, caused for example by the use of PPI medication. There is thus a need to find alternative ways to bind these aldehydes in the stomach in a harmless manner.

### BRIEF DESCRIPTION OF THE INVENTION

It is an aim of the present invention to provide new compositions, which can be used to reduce the aldehyde content in the stomach. It is also an aim of the present invention to provide new methods for binding aldehydes in the stomach.

Particularly, it is an aim of the present invention to provide new compositions, which protect the active compound(s), for example to mask their taste or to prevent their immediate release.

These and other objects, together with the advantages thereof over known compositions and methods are achieved by the present invention, as hereinafter described and claimed.

Thus, the present invention concerns a non-toxic composition containing one or more cysteine compounds for decreasing the risk of a subject contracting cancer of the stomach, and indirectly of the small intestine and the large intestine, by locally decreasing the content of aldehydes present in the stomach, and optionally also decreases the formation of these aldehydes.

The composition is capable of binding aldehyde present at least in the stomach, and comprises one or more aldehyde-binding compounds (such as cysteine or a derivative thereof), which are bound at least to such non-toxic additives that effect sustained release of said active compound(s) into the stomach.

Particularly, the composition according to the invention is characterized by what is stated in the characterizing part of claim 1.

The invention provides considerable advantages. The compositions comprising aldehyde-binding compounds can be used to reduce the risk of developing the cancer of the stomach, the intestine and/or colon of people having increased risk for cancer in these areas. By the compositions and methods of the invention can be treated in particular people suffering from atrophic gastritis, achlorhydric and low acid stomach, specifically when administered together with medication that causes a decreased acid-formation in the stomach. In these individuals, acetaldehyde is produced locally by mouth-derived bacteria that are able to survive in the neutral environment of the stomach in that they metabolize alcohol or sugars to acetaldehyde.

Furthermore, the compositions of the present invention are effective for binding aldehyde, in particular, when they are consumed in connection with eating, or when they are consumed in connection with consuming alcohol.

The same is true for smoking or other ways of using tobacco, i.e. the compositions of the present invention are particularly effective and particularly useful for binding aldehydes when they are consumed in connection with smoking or other ways of using tobacco.

Consuming the compositions according to the invention mainly binds aldehydes locally, due to the local release in the desired areas of the gastrointestinal tract (the stomach), but it also has a systemic effect.

### BRIEF DESCRIPTION OF THE DRAWINGS

FIG. 1 shows the results of a dissolution test using capsules according to the present invention, containing L-cysteine that is released at a controlled rate, yet fast enough to have time to react with acetaldehyde before leaving the stomach.

FIG. 2 shows the effect of L-cysteine administration (or placebo administration) on acetaldehyde levels.

FIG. 3 shows the mean cysteine concentrations in the gastric juice of volunteers after the administration of study formulations containing L-cysteine.

### DETAILED DESCRIPTION OF THE EMBODIMENTS OF THE INVENTION

The present invention concerns a non-toxic composition containing one or more aldehyde-binding compounds, preferably being cysteine compounds, such as L- or D-cysteine, N-acetyl cysteine, and the pharmaceutically acceptable salts thereof, and optionally one or more further active compounds.

Said further active compounds can be selected from sulphites, which are capable of binding aldehydes, and xylitol, which is capable of inactivating aldehyde-forming microbes carried to the stomach.

The composition is used for decreasing the risk of a subject contracting cancer of the stomach, and indirectly of the small intestine and the large intestine, by locally decreasing the content of aldehydes present in the stomach. Thus, the active compounds of the composition can include (in addition to aldehyde-binding compounds) also compounds reducing the formation of aldehydes. However, all these active compounds contribute in reducing the final content of aldehydes in the stomach.

The composition of the invention is formulated with the help of two or more additives into a controlled-release formulation consisting of granules containing at least a portion of said active compounds, the granules being contained in a capsule. At least one of the additives thus forms the capsule, while at least one further additive functions as a binder in the granules.

The active compounds are used in a pharmaceutically effective amount, which for the aldehyde-binding compound(s) means an amount capable of binding or inactivating the amount of aldehyde carried to or formed in the gastrointestinal tract of a subject during the consumption of food or drinks, or during the smoking of tobacco products.

At least one of the aldehyde-binding compound(s) is selected from cysteine compounds, preferably from L- or D-cysteine or the derivatives or pharmaceutically acceptable salts thereof. A particularly preferred derivative of cysteine is N-acetyl cysteine. Other alternatives can be:

cystine,
cysteic acid,
cysteine glycine,
threo or erythro-β-phenyl-DL-cysteine,
β-tetramethylene-DL-cysteine,
D-penicillamine and its dipeptides with N-terminals,
peptide or a protein with terminal cysteine,
glutathione,
reduced glutathione,

### D,L-homocysteine,

D,L-homocysteic acid,

### L-cysteinyl-L-valine,

β-β-tetramethylene-DL-cysteine,
cysteinyl-glycine,
tre-(5)-β-phenyl-DL-cysteine,
erythro-β-phenyl-DL-cysteine, and
cysteine hydrochloride.

Typically, a single unit, or formulation, of the composition comprises 50-500 mg, preferably 50-300 mg, more preferably 100-250 mg, and most suitably 100-200 mg of the cysteine compound(s). However, 1-2 of these units can be administered at once.

The content of the cysteine compound(s) is then 1 to 40 w-%, preferably 5 to 40, more preferably 10 to 30 w-%. Typically the amount is 20 to 25 w-%.

In case the composition contains xylitol as a further active compound, its content is preferably 10-90%, more preferably 10-60%, particularly 20-60%, and most suitably 40-60% of the weight of the composition. Typically, the content is about 50%.

Sulphites, which are another type of optional further active compounds, are generally used in smaller contents, which can be 50-400 ppm, preferably 50-300 ppm, and most suitably 50-200 ppm.

The composition of the invention comprises at least one non-toxic additive that effects sustained release of the aldehyde-binding compound(s) in the stomach. Sustained release means the release of said aldehyde-binding compound(s) for at least 30 minutes in the conditions of the stomach. Preferably the compound(s) are released during 0.5 to 8 hours, more preferably 2 to 6 hours, most preferably 2 to 4 hours. The capsule causes some delay in the release, which depends on, among others, the material and diameter of the capsule. However, it is preferred to use also other additives delaying the release.

The term “additive” here includes carriers, fillers, binders, granule coatings, as well as aromatic agents, colorants and non-functional additives. These additives are non-toxic, and preferably at least a portion of them function by controlling the release of the active compound(s) to take place specifically in the stomach, and most suitably in a sustained manner. Another objective of using said additive(s) is to protect the active compound(s), to mask their taste.

According to an aspect of the invention, at least one of the additives (e.g. a polymeric additive) forms said capsule, into which the remaining constituents of the composition are added. Preferably, such a capsule is filled with the remaining constituents in granulated form, whereby each granule can optionally be covered with a further polymeric film.

Said granules can be prepared by moisturizing a dry mixture of the constituents of the composition and granulating it by using methods and devices that are well-known in the pharmaceutical industry.

It is of further advantage if the composition is in the form of a capsule, the diameter of which is at least 7 mm, preferably 8 to 15 mm, more preferably 11 to 15 mm. This assists the unit to stay undissolved in the stomach for a sufficient time to cause the sustained release of aldehyde-binding compound(s).

Preferably the size of the capsule and the additives are selected to achieve the release of cysteine compound(s) in the conditions of the stomach in an amount of 40-80 mg in an hour.

For example, two L-cysteine molecules readily react with each other to form cystine (by dimerizing) that is not able to effectively bind and inactivate the aldehyde present in the stomach, particularly when used alone in a conventional immediate-release dosage form. However, the present invention provides a dosage form (particularly a capsule to be swallowed by the subject) that gives a long-term effect, and prevents said dimerization. Furthermore, acetaldehyde is produced during the entire time that the food stuff and/or alcohol resides in the stomach. Thus, the said formulation provides sustained source of L-cysteine over the expected time of acetaldehyde exposure.

Preferably, the used additives are selected from those capable of controlling the release of the active compound(s) so that these compounds are released locally in the stomach during a time of more than 30 minutes, preferably 0.5-8 hours, most suitably in 2-4 hours.

According to a preferred embodiment of the present invention, the composition is administered in connection with eating, i.e. just before, during or just after eating, or in connection with consuming alcohol, i.e. just before, during or after consuming a dose of alcohol.

According to another preferred embodiment of the invention, the composition is administered in connection with smoking or other use of tobacco, i.e. just before, during or just after smoking (or other use of tobacco).

A further advantageous option is to administer one dose (e.g. one unit) of the composition just before and another dose just after eating, drinking or smoking.

The terms “just before” and “just after” mean a time frame of up to 5 minutes before or after eating, consuming alcohol or smoking (or otherwise using tobacco), preferably a time frame of up to 2 minutes, more preferably a time frame of up to 1 minute, and most suitably a time frame of up to 0.5 minutes before or after eating, consuming alcohol or smoking.

However, the compositions can also be used in a continuous way, for example every 10 minutes. According to a preferred embodiment of the invention the dosage is renewed at 5-to 15-minute intervals, preferably at 5- to 10-minute intervals, if alcohol consumption or smoking is continued for an interval longer than the said one. Alternatively, the composition is administered at 4- to 10-hour intervals, preferably at 6- to 8-hour intervals.

“Smoking” refers to the smoking of any tobacco product, using snuff, chewing tobacco, or any other use of a tobacco product, wherein the tobacco product or a part thereof is placed in the mouth or in close vicinity to the oral cavities. The tobacco product can thus be a cigarette, a cigar, snuff, chewing tobacco or pipe tobacco.

The “binding of aldehyde” preferably refers to a chemical reaction between the aldehyde and the free sulphhydryl or amino group or both of the cysteine (or similar compound), wherein the aldehyde jointly with the “aldehyde-binding compound” forms a larger molecule. In the reaction with cysteine, for example acetaldehyde mainly binds itself to the sulphhydryl and the amino group of the cysteine, and forms 2-methyl-L-thiazolidine-4-carboxylic acid (and water).

According to the invention, the compounds obtained from aldehydes by chemical binding with cysteine are safe for the subject.

However, the aldehydes (in free form) are not harmless for the subject. A harmful/carcinogenic content of acetaldehyde in e.g. the human mouth is roughly 20 to 800 μmol/l of saliva, and a content of as low as about 20 to 50 μM causes carcinogenic DNA adducts on the cell level. Generally, levels of above 40 to 100 μM are considered mutagenic. Further, formaldehyde is responsible for some carcinogenic effect, while acrolein causes other toxic effects. Since the saliva that is carried to the stomach is also the main cause of the aldehyde-contents of the stomach, similar values are valid also for the present invention.

By administering the composition of the invention, the aldehyde content in the stomach can be reduced to a level that is essentially lower than without the use of the composition, which means that the aldehyde content can be kept at a level that is at least 20% lower, preferably >40% lower, and most suitably >60% lower than in a corresponding situation without using the composition according to the invention.

Such a harmful or carcinogenic content of aldehyde in the human stomach, as well as in the other parts of the gastrointestinal tract, can be caused by consuming alcoholic drinks, particularly strong alcoholic drinks, or foodstuffs containing alcohol, or as a consequence of smoking, or when consuming products (e.g. foodstuffs) containing aldehyde.

The aldehydes can be formed from the ethanol generated by oral microbes. Both these microbes and the formed aldehydes are constantly carried to the stomach with the saliva, particularly as the subject swallows. The “oral microbes” are intended to include oral bacteria and microbes in the oral cavity, such as streptococci, lactobacilli, corynebacteria, oral spirochetes, anaerobic cocci, and specifically Porphyromonas gingivalis, and various Candida species, including C. glabrata, C. parapsilosis, C. tropicalis, C. dubliniensis, C. guilliermondii, C. albicans, and C. krusei (in the pharynx). However, the aldehyde can also be contained in the alcoholic drink or in the foodstuff, either as a product of the manufacturing process, i.e. being a fermentation product, or it can be added as such into the drink or the foodstuff.

“Alcoholic drinks” are ethanol-containing drinks, their ethanol content varying within 0.7% by volume and 84% by volume.”

“Alcoholic foodstuffs” refer to foodstuffs containing at least 0.7% by weight of ethanol. Such foodstuffs can be, for example, fermented juices, yoghurts, any pickled food or other preserves, or foodstuffs preserved with small amounts of alcohol, pastries, jellies, and mousse seasoned with liqueur or corresponding products containing alcohol.

“Aldehyde-containing foodstuffs” refers to foodstuffs containing aldehyde even prior to consumption (i.e. in contrast to the aldehyde formed in the mouth of a subject consuming said foodstuff). Among others, acetaldehyde can be formed in said foodstuffs from ethanol that is generated in connection with fermentation, such as in beer, cider, wine, home-brewed beer, and other alcoholic drinks, as well as in many juices. In certain foodstuffs, such as some dairy products, this acetaldehyde is used for preservation purposes and to add flavour, or the acetaldehyde is formed in the product as a consequence of microbial activity. For example, sugary juices or sugar-containing foodstuffs, in general, provide a suitable substrate for acetaldehyde-producing microbes. High concentrations of acetaldehyde are also formed in fermented dairy products, such as yoghurt. In that case, mainly the microbes used to make yoghurt produce the acetaldehyde. As for alcoholic drinks, sherry and Calvados contain also especially large amounts of acetaldehyde.

The use of the compositions according to the invention can be of benefit even, when light alcoholic drinks or foodstuffs are consumed, i.e. those containing only small amounts of alcohol (even <0.7%), since even these contents are carcinogenic in the long run.

The additive(s) in the composition include a combination of substances, which can function specifically as carriers, fillers, binders, granule coatings and other types of additives.

According to a preferred embodiment of the invention the composition comprises at least one additive that does not dissolve or dissolves only poorly in the stomach. One option for achieving this is to cover the granules with an essentially water-insoluble film, or with a capsule.

According to another preferred embodiment of the invention, the composition comprises at least one additive selected from those that forms a gel in the conditions of the stomach, which gel then helps to maintain the components of the composition floating in the contents of the stomach for a prolonged time. Such a gel-forming additive can be added into a dry mixture to be granulated, or it can be added separately, with the pre-formed granules into the capsule.

Suitable additives not dissolving in the stomach include polymers, such as a hydroxypropylmethyl cellulose, polypropylene, Carbopol, or metacrylate polymer, for example Eudragit RL, RS, NE or S, or ethyl cellulose, and generally have a function that is based on their insolubility and on their swelling. Additives for use in achieving a gel are various chitosans, alginates, such as sodium alginate, aluminium hydroxide, sodium hydrogen carbonate, sodium carboxymethyl cellulose, and sodium hydrogen carbonate as described in WO 02/36098.

It is particularly preferred to achieve a formulation that floats in the contents of the stomach by using polymers, such as alginic acid, as additives, which form a gel, or inherently have a lower density than the aqueous contents of the stomach, and thus allowing flotation.

A gel that floats in the stomach can also be prepared from sodium alginate, aluminium hydroxide, sodium hydrogen carbonate, and water, to which the aldehyde-binding compound(s), or the granules formed thereof, can be added.

A corresponding formulation is also obtained by adding the aldehyde-binding compound(s), or the granules formed thereof, to an aqueous dispersion of chitosan.

The content of said polymers, or other gel-forming agents, in the composition is preferably 10-50 w-%, more preferably 20 to 40 w-%, and most suitably 20 to 30 w-%.

The additives of the composition can also include one or more bulking agents or fillers, preferably selected from calcium hydrogen phosphate, microcrystalline cellulose (MCC), lactose, or other corresponding bulking agents that are either water-soluble or non-soluble in water. Particularly, the bulking agents are selected from non-swelling agents. These are preferably mixed with the active compound(s) before granulating, whereby the bulking agent ends up inside the granules.

The content of such optional bulking agents in the composition is preferably 20-70 w-%, more preferably 40 to 60 w-%, and most suitably about 50 w-%.

Further, the additives of the composition can include one or more aromatic agents, such as flavourings, particularly included in the material forming the capsule. Typical aromatic agents include carbohydrates (or sugars), such as glucose, sorbitol, eucalyptol, thymol, sucrose, sodium saccharine, methyl salicylate, menthol and xylitol, and are preferably selected from glucose, sorbitol, sucrose and xylitol. However, as stated above, xylitol can also be present as an active compound, whereby at least a major part of it will be included in the contents inside the capsule.

As stated above, the composition is formulated by forming a capsule that contains the remaining constituents of the composition. The capsule is preferably formed from hydroxypropyl methyl cellulose (HPMC) or gelatine, such as hard gelatin, and is most suitably formed from HPMC. At least a major part (such as >50% by weight) of the remaining constituents are added into the capsule in granulated form.

According to a preferred embodiment, this is achieved by mixing said remaining constituents into a dry mass, and granulating by using enteric polymers as binders. These are preferably selected from polymers with a solution pH of 6-7, such as hydroxypropylmethyl cellulose, polypropylene, Carbopol, or methacrylate, and most preferably from methacrylate derivatives, which are known by the trade names Eudragit L, Eudragit S, and Eudragit RS. The amount of enteric polymer in the preparation is preferably 2-5%, most preferably 3-4%.

The granules can optionally be coated with a further polymeric film. Such a polymeric film can be formed using porous film forming agents, such as ethyl cellulose (EC) and hydroxypropyl methylcellulose (HPMC). Preferably a mixture of these are used, more preferably a mixture, where the relative amount of EC to HPMC is 1/1 to 5/1, preferably 2/1 to 5/1, and most suitably 3/2 to 7/3.

Such a mixture has advantageous dissolution properties due to the different characters of the constituents, as HPMC is a water-soluble polymer and EC is a water-insoluble polymer. In the conditions of the stomach the water-soluble polymer dissolves and pores are formed to the water insoluble polymer. In such a case, the release of the highly water-soluble cysteine compound(s) is based on its diffusion from the pores formed to the film. Such film-forming substances also effectively mask also the taste of the cysteine compound(s).

An exemplary composition in the form of a capsule comprising granules can have the following contents:

| | |
|---|---|
| Aldehyde-binding substances | 20-40 parts |
| Enteric polymer | 10-40 parts |
| Calcium hydrogen phosphate | 20-60 parts |

in granulated form, wherafter these granules are added into an HPMC capsule.

In addition to the above active compounds and additives, it is of advantage to add to the compositions of the present invention at least one of the substances selected from the group comprising chromium, vitamin B12, A-, D-, E, -C-vitamins, niacin, biotin, thiamine, B2-, B5-, B6-vitamins and folic acid and trace elements, such as chromium, manganese, selenium, zink and iron.

According to one preferred embodiment of the invention the composition of the present invention is administered to people having an achlorhydric stomach in connection with administering medication that causes said achlorhydric stomach, such as proton pump inhibitors.

One component of gastric juice is hydrochloric acid (HCl), the secretory product of the parietal, or oxyntic cell of the corpus of the stomach. It is known that the capacity of the stomach to secrete HCl is almost linearly related to parietal cell numbers (Yao et al. 2003, Samuelson et al. 2003). Acid secretion is dependent on function of the H+/K+ATPase or proton pump located in the cannilicular membrane of the parietal cell.

Several drugs have been developed that non-competitively bind and inactivate the ATPase, resulting in strong inhibition of acid secretion. Such drugs include proton pump inhibitors (PPIs), such as:
Dexlansoprazol
Esomeprazol
Lansoprazol
Omeprazol
Pantoprazol
Rabeprazol
Tenatoprazol

For example, omeprazole (such as Prilosec) is an acid-activated prodrug that binds covalently to two cysteines on the ATPase, resulting in its irreversible inactivation. Other proton pump inhibitors (PPIs), including lansoprazole (Prevacid), esomeprazole (Nexium), rabeprazole (Aciphex) and pantoprazole (Protonix) have similar modes of action (Hellstrom et al. 2004, Sachs et al. 1994, Shamburek et al. 1992, Welag et al. 2003).

According to a particularly preferred embodiment of the present invention, the composition includes (in addition to the aldehyde-binding compounds), one or more of said PPIs, whereby a combination product is obtained that is intended for ameliorating the symptoms of a hyperchlorohydric stomach, an H. pylori infection, gastroesophageal or oesophageal reflux disease or atrophic gastritis.

The following non-limiting examples demonstrate the advantages obtained with the preferred embodiments of the invention.

### EXAMPLES

### Example 1

### Preparation of Capsules According to the Present Invention

The capsules were prepared by mixing 500 g of L-cysteine (Gonmisol S.A., Spain), 500 g of Eudragit RS-PO, forming a matrix structure (Evonik Röhm GmbH, Germany), and 1 kg of calcium hydrogen phosphate (Emcompress® Anhydrous; Mendell a Penwest Company, Lakeville, Minn.) in a Turbula Powder Blender (Glen Mills Inc., Clifton, N.J.) for 10 minutes.

The mixture was wet-granulated using ethanol. The obtained wet granules were sieved using a 2-mm sieve, and thereafter allowed to dry at room temperature in a fume hood for 24 hours. The dried granules were sieved using a 1.68 mm and 1.18 mm sieves, and the obtained middle fraction was collected for capsulation.

Simultaneously, a placebo formulation, where the L-cysteine was replaced by the same amount of CaHPO₄, was prepared following the exact same procedure.

The obtained matrix granules were weighed into HPMC capsules so that the desired amount of cysteine per capsule was obtained. The L-cysteine concentration of the granules was determined using a capillary method (400 mg of granules contained 98 mg of L-cysteine). The amount of L-cysteine per capsule was left at 50 mg in order to ease the selection of a suitable dosage (for a dosage of 100 mg or 200 mg of L-cysteine, 2 or 4 capsules were administered at essentially the same time to the subject).

Similar capsules containing also titanium dioxide were prepared, and this excipient was found not to have an effect on the desired function of the capsule.

The capsules prepared above are ingested to decrease the risk of cancer locally caused by acetaldehyde in occasions, which are favourable for an increase in the acetaldehyde content of the stomach, such as in connection with consuming alcoholic drinks. The dosage is given at 4 to 6-hour intervals as long as there is alcohol in the blood.

### Example 2

### Dissolution Test for the Capsules

Dissolution tests were carried out on the capsules of Example 1 according to the USP I method (USP 24) (The United States Pharmacopeia 2001). A standard curve was prepared between 0.01 and 0.6 mg/ml (y=2.196+0.0016, r²=0.9999). The medium used was 500 ml of pH 1.2 HCl buffer. The rotation rate of the baskets was 100 rpm, and the temperature of the medium was +37° C. (±0.5). Samples were taken at 5-minute intervals for the first half hour and thereafter at 10-minute intervals for the remaining 2 hours. L-cysteine was detected in flow-through cells (10 mm) at a wavelength of 213 nm. The results were calculated by using dissolution software. The system was equipped with a bath and a pump (Sotax AT7 UV Dissolution System, Stax, Allschwil, Switzerland) and a spectrophotometer (PerkinElmer, Lambda 25, PerkinElmer, Inc., Waltham, Mass.), the software used for the test and for calculating the results was WinSotax (Sotax).

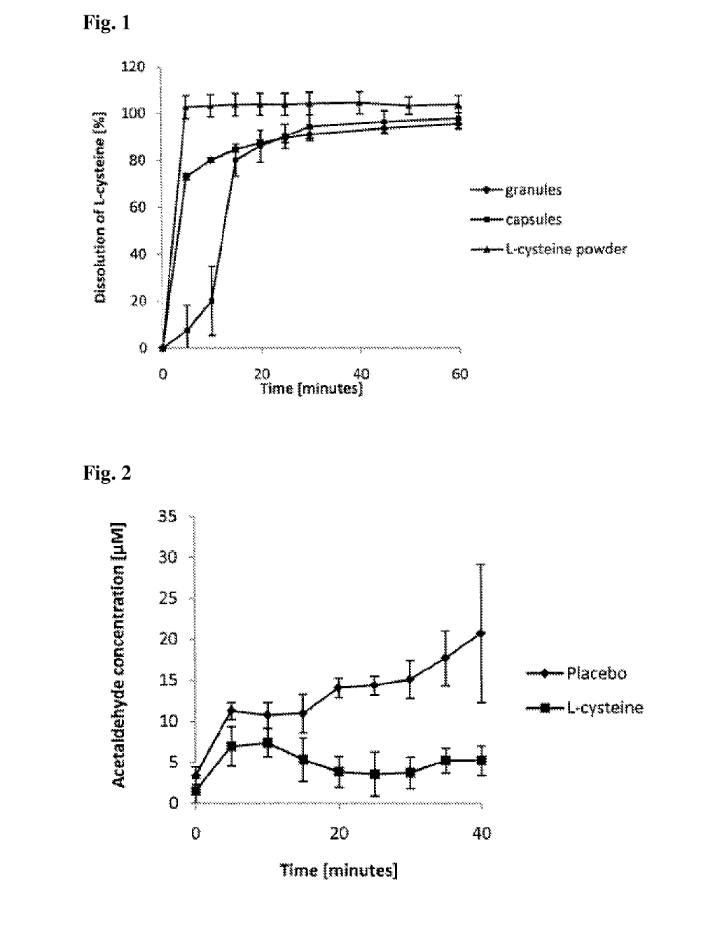

This dissolution test showed that the formulation released L-cysteine at a controlled rate, yet fast enough to have time to react with acetaldehyde before leaving the stomach. These results are shown in FIG. 1. When not granulated, the L-cysteine was dissolved rapidly (100% in 5 minutes).

### Example 3

### Acetaldehyde-Binding

### Study Procedure:

Seven volunteers (2 men, 5 women) with achlorhydric atrophic gastritis participated in the study. Their mean age±SD was 57±7 years and mean body weight 75±22 kg. All volunteers were non-smokers and normal social drinkers, with an average consumption of 50 g or less of ethanol per week.

A randomized double-blinded placebo-controlled study design was used, and each participant served as his/her own control. The 2 study days were separated by at least a 3-day interval. The volunteers were told to refrain from alcohol intake for 24 hours and food intake for 12 hours prior to the study.

A nasogastric tube (Duodenal tube Levin, CH10, Unomedical, Denmark) was inserted into the subjects to a depth of 55 cm at the beginning of each study day. The tube was lubricated with Xylocain gel (AstraZeneca, Sodertalje, Sweden) containing no ethanol. During the tube placement, the volunteers were given 100 ml of water to facilitate swallowing of the tube.

The subjects were given four capsules, containing either cysteine (50 mg in each capsule) or placebo, as prepared according to Example 1, orally double blindly with 200 ml of water. Immediately thereafter, ethanol (0.3 g/kg body weight) diluted in water to 15 vol %, was infused via the nasogastric tube into the stomach of the volunteers.

Samples of gastric juice (5 ml) were aspirated through the tube at 5-minute intervals up to 60 minutes after the ethanol infusion or until the stomach had emptied, as indicated by unsuccessful aspiration. The samples were analyzed for pH and acetaldehyde, ethanol and cysteine concentrations.

### Analysis:

To measure the acetaldehyde concentration, 450 μl of gastric juice was immediately transferred into a headspace vial containing 50 μl of 6 mol/1 perchloric acid. Perchloric acid does not hydrolyze the cysteine-acetaldehyde bond.

For the ethanol analysis, the gastric juice was diluted 10-fold in purified water, and 500 μl of diluted gastric juice was transferred into a headspace vial.

Two parallel samples were used for the measurements, and the mean value was calculated. The levels of acetaldehyde and ethanol were analyzed by headspace gas chromatography, as previously described (Vakevainen et al., 2002).

L-cysteine concentration of the gastric juice samples were determined by using an HPLC method. A standard curve was prepared between concentrations of 0.0625 and 2.0 mg/ml (y=851.06x+8.52, r²=0.9704). Two parallel samples were again prepared. 60 μl of gastric juice was measured into a test tube, and 30 μl of pH 7.4 phosphate-buffered saline solution and 30 μl of 20 vol-% tri-n-butyl phosphine in dimethylformamide were added. The samples were incubated for 30 minutes at +4° C., after which 90 μl of cold 10% trichloroacetic acid containing 1 mM Na₂EDTA was added, and the samples were vortexed for 2 minutes and then centrifuged for 10 minutes at 4500 rpm. 50 μl of supernatant was pipette into a test tube containing 125 μl of pH 9.5 borate buffer with 4 mM Na₂EDTA, 10 μl of 1.55 M sodium hydroxide, and 50 μl of 2 mg/ml 4-fluoro-7-sulfobenzofurazan, ammonium salt (SBD-F) solution in borate buffer. The samples were incubated for 60 minutes at +60° C. so that a yellow derivate was formed. Thereafter, 150 μl of the solution was pipette into HPLC inserts, and used for the measurements. The isocratic mobile phase was pH 7.0 phosphate buffer and methanol (95:5). The flow rate was 1 ml/min and the retention time was 6 minutes. The L-cysteine concentration was determined using a fluorescence detector (excitation 385 nm, emission 515 nm).

### Results:

FIG. 2 shows the effect of the L-cysteine administration (or the placebo administration) on the acetaldehyde levels. In all measurements, the average acetaldehyde concentration of the gastric juice was 2.6 times higher with placebo than with cysteine. No significant differences existed in ethanol concentrations between cysteine and placebo treatments. The average ethanol concentration in the gastric juice was 5.0 vol-% in the first sample, declining to 0.9 vol-% in the 40-minute sample. A positive correlation emerged between the acetaldehyde concentration and the ethanol concentration.

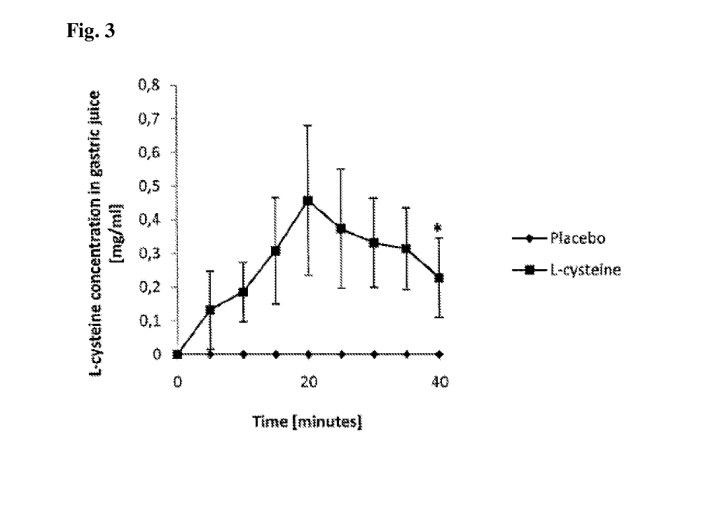

L-cysteine was detected in the gastric juice of all volunteers after the administration of study formulations containing L-cysteine. The mean cysteine concentrations are represented in FIG. 3. After administration of placebo formulations, no L-cysteine was detected. No significant correlation was found between the cysteine concentration and the acetaldehyde concentration.

### LITERATURE

Francheschi et al. Cancer Res 1990; 50:6502-07
Haussmann, H.-J. Chem. Res. Toxicol. 2012; 25, 794-810
Hellstrom P M, Vitols S: The choice of proton pump inhibitor: does it matter? Basic Clin Pharmacol Toxicol 2004; 94:105
Homann et al, Carcinogenesis 1997; 18; 1739-1743
International Agency for Research on Cancer, 1999; Acetaldehyde. In: Re-evaluation of some organic chemicals, hydrazine and hydrogen peroxide. IARC Monographs on the evaluation on the Carcinogenic Risks to Humans, vol 71, pp. 319-335.
Jokelainen et al. Gut 1996; 39:100-104
Sachs G, Prinz C, Loo D, etc: Gastric acid secretion: activation and inhibition. Yale J Biol Med 1994; 67:81-95
Salaspuro, M. Best Pract Res Clin. Gastroenterol 2003; 17:679-94
Salaspuro M. Acetaldehyde as a common denominator and cumulative carcinogen in digestive tract cancers. Scand J Gastroenterol 2009; 44: 912-925.
Samuelson L C, Hinkle K L: Insights into the regulation of gastric acid secretion through analysis of genetically engineered mice. Annu Rev Physiol 2003; 65:383-400
Secretan B, Straif K, Baan R, Grosse Y, El Ghissassi, Bouvard V et al. A review of human carcinogens—Part E: tobacco, areca nut, alcohol, coal smoke, and salted fish. Lancet Oncol 2009; 10: 1033-1034.
Seitz H K, Stickel F. Acetaldehyde as an underestimated risk factor for cancer development: role of genetics in ethanol metabolism. Genes Nutr 2010; 5: 121-128.
Shamburek R D, Schubert M L: Control of gastric acid secretion. Histamine H2-receptor antagonists and H+K(+)-ATPase inhibitors. Gastroenterology Clinics of North America 1992; 21:527-550
Väkeväinen et al. Scand. J. Gastroenterol 2002; 37:648-655
Welage L S: Pharmacologic properties of proton pump inhibitors. Pharmacotherapy 2003; 23:74S-80S
Yao X, Forte J G: Cell biology of acid secretion by the parietal cell. Annu Rev Physiol 2003; 65:103-131

1. A non-toxic composition containing, as active compounds, one or more cysteine compounds selected from the group of L- or D-cysteine, N-acetyl cysteine, and the pharmaceutically acceptable salts thereof, for decreasing the risk of a subject contracting cancer of the stomach, and indirectly of the small intestine and the large intestine, by locally decreasing the content of aldehydes present in the stomach,
characterized in that
the composition is formulated with the help of two or more additives into a controlled-release formulation consisting of granules containing one or more active compounds, the granules being contained in a capsule, whereby at least one additive forms the capsule and at least one additive functions as a binder in the granules, and
optionally, in that the composition further contains one or more further active compounds selected from sulphites, capable of binding aldehydes, and xylitol, capable of inactivating aldehyde-forming microbes carried to the stomach.

2. The composition according to claim 1, which is intended for administration to a subject in connection with eating, in connection with drinking alcoholic drinks, or in connection with smoking to reduce the risk of developing cancer of the area of the stomach.

3. The composition according to claim 1, which comprises cysteine compound(s) in an amount of 50-500 mg, preferably 50-300 mg, more preferably 100-250 mg, and most suitably 100-200 mg per unit dose.

4. The composition according to claim 1, which releases the active compounds during a time period of more than 30 minutes, preferably 0.5-8 hours, most suitably in 2-4 hours, after administration.

5. The composition according to claim 1, wherein the capsule is formed of hydroxypropyl methyl cellulose (HPMC) or gelatine, such as hard gelatin, and is most suitable formed of HPMC.

6. The composition according to claim 1, wherein the binders are selected from polymers, such as hydroxypropylmethyl cellulose, polypropylene, Carbopol or methacrylate, preferably polymers with a solution pH of 6-7, and most preferably from methacrylate derivatives, which are known by the trade names Eudragit L, Eudragit S, and Eudragit RS.

7. The composition according to claim 6, wherein the amount of binding polymer is 2-5%, preferably 3-4%.

8. The composition according to claim 1, wherein the granules are separately coated with a polymeric film formed using porous film forming agents, such as ethyl cellulose (EC) and hydroxypropyl methylcellulose (HPMC), preferably a mixture of these, more preferably a mixture, where the relative amount of EC to HPMC is 1/1 to 5/1, particularly 2/1 to 5/1, and most suitably 3/2 to 7/3.

9. The composition according to claim 1, which contains at least one additive selected from cationic and gel-forming polymers, selected from matrix-forming polymers, such as methacrylate polymers, hydroxypropylmethyl cellulose, polypropylene, Carbopol, ethyl cellulose, sodium carboxymethyl cellulose, chitosans, and alginates, preferably from the methacrylate derivatives Eudragit L, S, RL, RS or NE.

10. The composition according to claim 9, which further includes one or more non-polymeric gel-forming additives selected from aluminium hydroxide and sodium hydrogen carbonate.

11. The composition according to claim 9, having a content of gel-forming additives of 10-50 w-%, preferably 20 to 40 w-%, and most suitably 20 to 30 w-%.

12. The composition according to claim 1, which contains at least one additive selected from polymers not dissolving in the stomach, preferably from Eudragit RS or S, or ethyl cellulose.

13. The composition according to claim 1, which contains one or more bulking agents, preferably selected from calcium hydrogen phosphate, microcrystalline cellulose (MCC), lactose, or other corresponding bulking agents that are either water-soluble or non-soluble in water.

14. The composition according to claim 13, having a content of bulking agents of 20-70 w-%, preferably 40 to 60 w-%, and most suitably about 50 w-%.

15. The composition according to claim 1, which further contains one or more proton pump inhibitors (PPIs) as active compounds, and the composition is intended for ameliorating the symptoms of a hyperchlorohydric stomach, an H. pylori infection, gastroesophageal or oesophageal reflux disease or atrophic gastritis.

16. A method for decreasing the risk of a subject contracting cancer of the stomach, and indirectly of the small intestine and the large intestine, by locally decreasing the content of aldehydes present in the stomach, wherein a non-toxic encapsulated composition containing one or more active compounds, at least one being selected from the group of L- or D-cysteine, N-acetyl cysteine, and the pharmaceutically acceptable salts thereof, is administered to the subject, which composition is formulated with the help of two or more additives into a controlled-release formulation consisting of granules containing one or more active compounds, the granules being contained in the capsule, whereby at least one additive forms the capsule and at least one additive functions as a binder in the granules.
